(19) United States
(12) Patent Application Publication
(10) Pub. No.: US 2007/0113861 A1
(43) Pub. Date: May 24, 2007

(54) Cap for a medical device

(75) Inventors: Thomas Kamp Knudsen, Copenhagen N (DK); Steve McGugan, Lyngby (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/593,374

(22) Filed: Nov. 6, 2006

(30) Foreign Application Priority Data
Nov. 8, 2005 (EP) 05024263.5

(51) Int. Cl. A61B 19/00

(52) U.S. Cl. 128/898; 604/263

(57) ABSTRACT

A protective cap for a medical device comprising two cap parts, an inner and an outer cap part, which two cap parts can be assembled, one inside the other, to form a single integrated cap. When assembled, the inner and outer cap parts have a gap in-between, where a user defined object can be inserted. Interlocking means are provided to hold the cap parts together, and seals can be provided to assure a tight enclosure. Elevations may be provided to locally narrow the gap between the two cap parts in order to hold the inserted object in place and a template may be provided to cut out the objects to be inserted in appropriate dimensions. The outer cap part may be transparent to make the inserted object visual, whereas the inner cap part may be impenetrable by light, to protect the medical device.

### CROSS-REFERENCE TO RELATED PATENT APPLICATIONS

This patent application claims priority of EP 05024263.5 filed Nov. 8, 2005 with the European Patent Office.

### TECHNICAL FIELD OF THE INVENTION

The invention relates to a protective cap for a medical device.

As most medical devices are designed to be brought in contact with the human body, or even to penetrate the human body, parts of the medical devices often need to be provided with a protective closure in releasable attachment to the device. The protective closure typically serves two purposes, namely to protect the medical device and its contents from ambient influences such as dirt, dust and light and also the protective closure serves to protect against unwanted contact between the human body and hazardous parts of the medical device, for instance needles or reactive medias.

This invention relates to an additional purpose of a protective cap for a medical device. When a medical device is provided with a protective cap as a necessity, it is of great advantage to provide for the cap to be personalized, even more so, if the cap personalisation could easily be modified by the user itself. Especially when the user is a child, the possibility of a personal influence on the appearance of the medical device can make the otherwise unpleasant and potentially frightening act of taking a medication less unfamiliar and a natural part of the child's everyday by diverting its attention from the actual medication act. Thus, when using the personalised protective cap according to this invention, especially children will be more motivated to follow their prescribed treatment. As will be described in more detail hereafter, the personalisation can also serve the purpose of providing information or colour codes.

An automatic needle insertion mechanism for syringes also deals with the user discomforts when taking a medication. Without limiting the scope of the present invention, it shall be noted that such an automatic needle insertion mechanism is a good example of potential use of the personalised protection cap as here described, as these are often used by people who look at the act of taking medication with disinclination.

### DESCRIPTION OF RELATED ART

From mobile phones the concept of different exchangeable covers is known. The covers are available in a variety of colours and patterns; however the covers can not be easily modified and personalised by the user.

WO 03/086511 describes a system for modification of an injection device. The system consists of two elements to be attached to the housing, one at a time. The two elements have different properties in at least one respect such as size, shape, functional properties, aesthetic or information carried. These two elements, however, are not a normal part of the device, but rather an “add-on” feature to the device making it more voluminous. Also the system does not make it possible for the user to easily personalize the device because of the attachment means which have to be of certain strength to fixate the elements to the housing. Neither do the two elements provide good protection of any sheets put between the elements while at the same time giving a well defined gap for housing of the sheet.

### DESCRIPTION OF THE INVENTION

The objective of the invention is to provide a protection cap for a medical device having means for easily and securely personalising a main part of the cap, without altering the shape of the medical device, in addition to the protection effect.

This is achieved by a cap of the kind described in the opening of this application, which cap according to the invention comprises two hollow cap parts, the first (inner) cap part designed to fit in the second (outer). The two cap parts are designed to releasable interlock to form one assembled unit, corresponding to a known protective cap. In one embodiment of the invention, at least the second cap part is made from a transparent material. The first cap part can in one embodiment be made impenetrable by light for protection of the medical device and the active media. Between the first and the second cap parts, a user defined object such as for instance a photograph, a drawing, both means for comforting the user, an address card, a pattern or a sheet containing printed or colour coded information can be inserted when the two cap parts are separated. After insertion of the object, the two cap parts are assembled and interlocked whereby the user defined object is contained in the gap between the first and the second cap parts. A prefabricated object such as a downloaded image from the internet or pre-cut sheets can also be contained in the gap between the two cap parts.

In an embodiment of the invention, the interlock is mechanical by means of a thread, a snap-lock, a bayonet lock, magnets, friction or paste. The threaded interlock can preferably be positioned in either end of the cap parts, thereby leaving the largest possible area free to display the inserted object. Especially when the interlock is of the thread or bayonet type, it can be an advantage if the two cap parts are cylindrical or conical as in one of the embodiments of the invention.

In an embodiment of the invention, a seal is provided between the two cap parts in the end of the cap provided with an opening to fit the medical device. The seal ensures a tight fit between the two cap parts preventing dirt, moist or other unwanted material from entering the gap between the two cap parts once the parts are assembled. This serves both an aesthetical purpose, but also a more practical purpose as the inserted object can be damaged if unwanted material, especially moist, enters the gap. Finally it is of great importance in a medical device to minimise any bacterial growth in any near presence of the device as this is a potential hazard to the user. The seal can be of any suitable material and design for instance o-rings, lib-seals, resilient material, adhesive materials, pastes or moulded baffles.

In a further embodiment, the first cap part has a protrusion at the end opposite to the opening of the protection cap. This protrusion fits through an opening or into a corresponding yet sufficiently bigger protrusion in the second cap part and serves the purpose of aligning the two cap parts and also it can be an advantage to position the interlocking means here: the first part of the interlocking means on the base of the protrusion on the transition between the protrusion and the first cap part, and the second part of the interlocking means in the opening for the protrusion or on the inside of the corresponding protrusion on the second cap part. This advantageous embodiment ensures not only tight and aligning interlocking of the cap parts, but also the maximum available display area for the inserted object. In this embodiment of the invention, it can be favourable to have a seal in the cap end with the protrusion as well as in the end with the opening for the medical device. In a further embodiment of the invention, the first cap part has at least one, preferably two radial protrusions located in the end of the first cap part having an opening to fit over the medical device. These radial protrusions serve two purposes. They protect the medical device from rolling, resulting in drop of the medical device and potential damage as a possible consequence, and also they serve to give a good support for the fingers when the user assembles or disassembles the two cap parts.

To ensure a correct positioning of the object inserted between the two cap parts, especially in the event of the inserted object having outer dimensions smaller than the maximum available area provided in the gap between the two cap parts, an embodiment of the invention has a variety of positioning and fixation means. The first cap part can be equipped with elevations on the outer side to locally narrow the gap between the two cap parts. Once the two cap parts are assembled, the elevations will secure the inserted object in the chosen position between the two cap parts. The elevations can in a preferred embodiment be dots, longitudinal ridges, helical ridges or circumferential elevations. The elevations can be made from a resilient and/or adherent material such as rubber or silicone or baffles, and to ease the assembly of the two cap parts the second cap part can have a funnel shaped inside in the end with the opening to fit over the medical device. In the case where the two cap parts are conical, the entire inside of the second cap part is funnel shaped, making the assembling of the cap parts easy and without risk of damaging the inserted object. A second solution to solve the problem of supporting the inserted object is an embodiment of the invention having a longitudinal or circumferential supporting edge or at least a part of the first cap part is provided with an adhesive material. The longitudinal or circumferential supporting edge can be of sufficiently large radial dimensions to locally close the gap between the two cap parts. In this embodiment, the edge not only serves as a supporting edge, but also divides the space between the two assembled cap parts into at least two compartments, axially or angularly displaced.

As mentioned, the problem of fixating the inserted object between the two cap parts especially arises when the outer dimensions of the inserted object is of smaller dimensions than the maximum available area in the gap between the two cap parts. Therefore in an embodiment of the invention, a template with outer dimensions corresponding to the largest size of a user defined object able to fit between the two cap parts is provided. When applying the template, the user can modify a given object to be put in the gap, either by holding the template over the object against a hard surface and with a knife cutting any surplus edges directly, or by drawing a line along the border of the template on the object and thereafter cutting off the surplus edges. The template can be of any material strong or hard enough to provide sufficient support to a knife without the knife cutting into the template. For instance the template can be made of metal or preferably of hard transparent plastic, thereby making it possible to view through the template and ensuring that the correct part of for instance any image, pattern or the like to be cut out is covered. By using the template, it is possible for the user to give a perfect appearance of the cap, even with inserted objects not provided as accessories to the cap, but freely chosen by the user.

The invention allows for personalising a cap for a medical device and thereby the medical device itself to a user's specific needs and wishes without altering the physical appearance of the device, thereby increasing the likelihood of the user following the prescribed treatment. The invention also solves the problem of having a tight enclosure for the inserted object to ensure the object is safe and no health hazard is introduced, while at the same time offering easy handling without the need for tools or great finger strength. Further the invention allows for a user to be able to freely select the object to be inserted while at the same time ensuring that the final appearance of the decorated cap is flawless with aligned, accurate fit and no unintentionally uncovered surfaces.

In a another aspect of the invention, the security of a medical treatment of a patient is increased by motivating the patient to comply with a correct use of a medical device by enabling the patient to interchange objects of hers/his choice in the cap of the medical device. This can serve to motivate the patient in a range of possibilities. In on aspect, the patient is rewarded when complying with a treatment or a correct use of a medical device, The reward can be objects to insert in the cap of the device. Especially when the patient is a child, it is particularly important that the patient is motivated, as the child not necessarily understands the importance of the medical treatment, or may not be fully mature to use the device correct or remember the correct moments for treatment. If the patient is rewarded with objects such as labels, pictures, patterns, figures, gaming pieces or toys when using the medical device correct, or complying with a treatment, there is a raised probability that the patient will indeed be in compliance. The objects can be elements of a series of objects, so the patient is always motivated to obtain the following object in the series. The object may also contain information, written, in pictures or in other ways visualized. This information can contain medication information, injection instructions, inhalation instructions, reminders, emergency telephone numbers, home page addresses, personal addresses, hospital or other emergency addresses, calendars and a further wide range of objects, only restricted by the physical borders of the given space in the cap. This possibility of storing information visible and always at hand near the medical device assures a more safe treatment of the patient as it is assured that instructions about the drug and/or instructions about treatment procedures are always close by: keeping the “manual” at hand decreases the risk of trial and error procedures being executed by the patient, as it is not cumbersome to check what is the correct measures. Further the risk of over-dosing is minimized if a calendar dedicated to the medical treatment is also always at hand, as the patient this way is motivated to easily keep track of the medical actions performed. If a critical situation arises however, it is a further advantage of this invention that emergency addresses or phone numbers are also easily viewable and available. To increase safety also a lock, tamper proof or at least means to make it evident that the storing means has been tampered with can be of advantage, as this makes it evident for the patient or medical professionals if the objects stored in the medical device, has been tampered with. In a further embodiment of the invention, the objects stored in the cap of the medical device can be objects able to sense and visualize surrounding climatic parameters. For instance the objects can change colour according to temperature, time or moist, either dynamically changing with the surrounding climate or statically visualizing an extreme value that the device has been exposed to, such as high or low temperature or moist, or time elapsed since activating the object (as indicator of how long the drug has been in use).

All headings and sub-headings are used herein for convenience only and should not be construed as limiting the invention in any way.

The use of any and all examples, or exemplary language (e.g. such as) provided herein, is intended merely to better illustrate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with various selected embodiments and with reference to the drawings in which:

FIG. 1 shows a side view of the first and second part of the cap when not assembled.

FIG. 2 shows a side view of the first part of the cap, where the cap part has a longitudinal or circumferential supporting edge to support an inserted object.

FIG. 3 shows a side view of the first cap part provided with circumferential seals.

FIG. 4 shows a side view of the first cap part provided with elevations.

FIG. 5 shows a template for an inserted object for the cap.

FIG. 6 shows an exploded, perspective view of the two cap parts and an object to be inserted in-between.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENTS

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as their relative dimensions are intended to serve illustrative purposes only.

In that context it may be convenient to define that the term “proximal end” in the appended figures is meant to refer to the end of the cap having the opening to fit over the medical device it is designed to protect, whereas the term “distal end” is meant to refer to the opposite closed end of the cap, enclosing an end of the medical device.

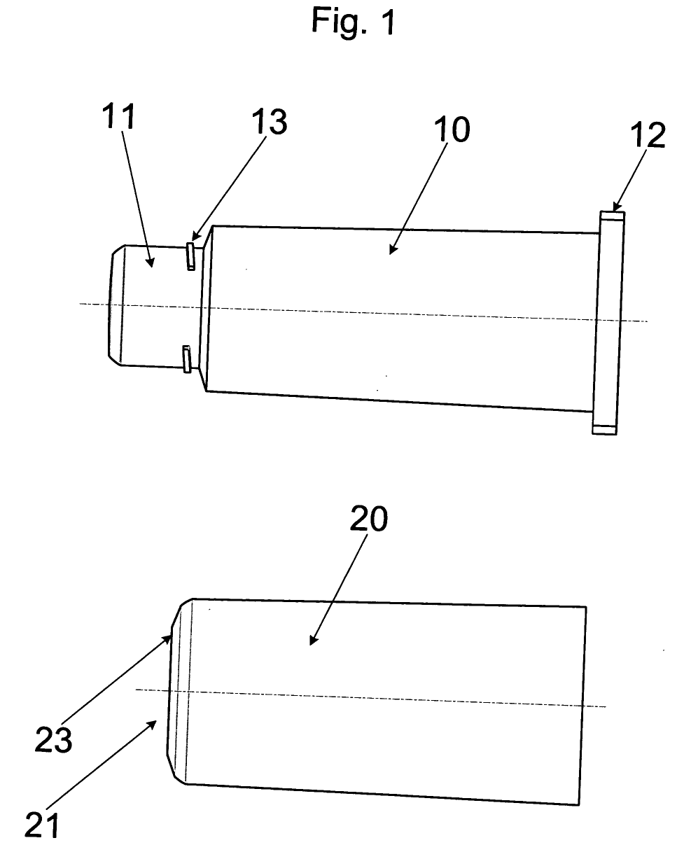

FIG. 1 discloses the two parts of the cap in an embodiment where both the parts are correspondingly conical. The first part 10 of the cap is provided with a protrusion 11 on its distal end to fit through a corresponding opening 21 in the second cap part 20. The two cap parts can be releasable interconnected, the first cap part 10 within the second cap part 20 with a gap in-between. This is obtained by means of an outer thread 13 provided on the protrusion 11 on the first cap part 10 and a corresponding inner thread 23 provided on the inner edge of the opening 21 in the second cap part 20. On the proximal end of the first cap part 10, there is a shoulder provided with two radial protrusions 12. These protrusions 12 serve the purpose of hindering the cap, and the medical device if the cap is mounted, from rolling when left on a surface, and also serve the purpose of giving the fingers a good grip on the cap when the cap is to be put on or off the medical device, or when the two cap parts are assembled or disassembled.

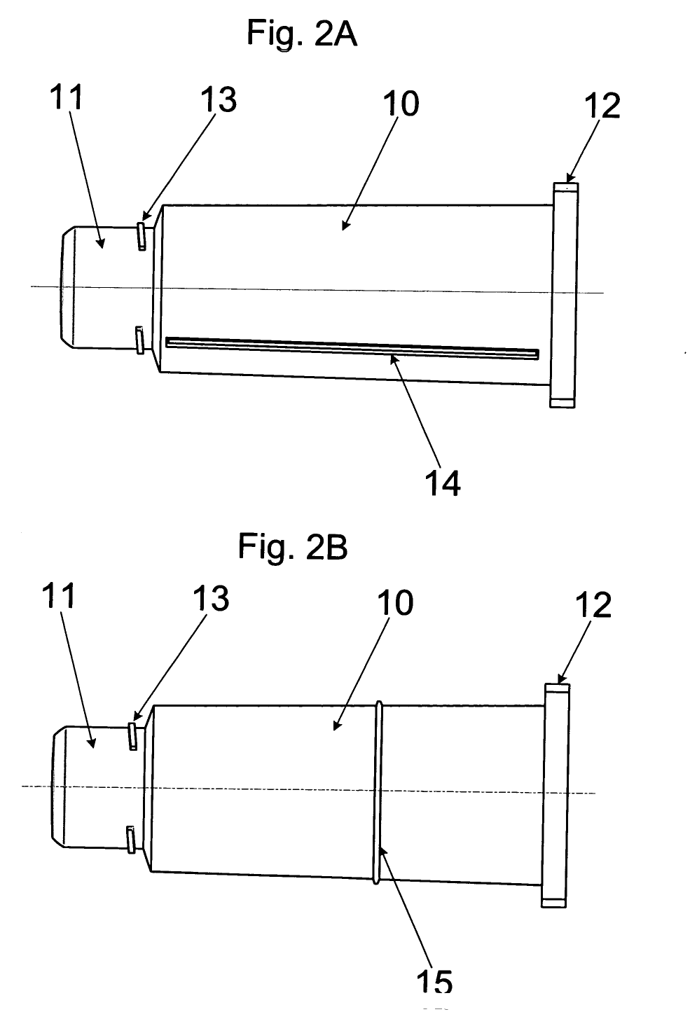

FIG. 2 shows an embodiment according to FIG. 1, but also provided with a longitudinal supporting edge 14, or a circumferential supporting edge 15 on the first cap part 10. This supporting edge serves to align and support a given object 30 inserted in-between the two cap parts. In the case of the circumferential supporting edge 15 shown on FIG. 2B, it also divides the space between the two cap parts in two.

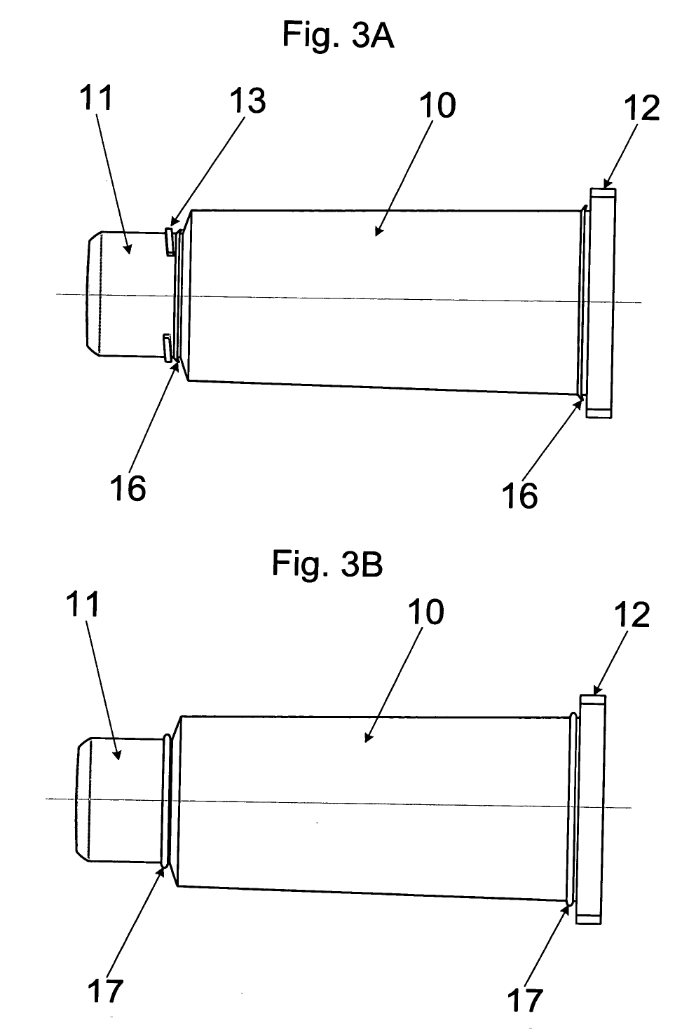

FIG. 3 discloses an embodiment of the invention where again, the cap parts are conical, and where the cap is provided with seals 16 or 17. When the outer cap part 20 has an opening 21, it is advantageous to have two seals, one in the proximal and one in the distal end of the cap, to ensure a safe and tight enclosure. On FIG. 3A an embodiment with lip seals 16 is shown, and on FIG. 3B an embodiment with a semi-round seal 17 is shown. In these embodiments the seals 16, 17 are provided on the first cap part 10. FIG. 3B also illustrates how the seals 17 can provide sufficient frictional resistance between the two cap parts 10, 20 and the seals 17, to render superfluous further interlocking means, as for instance the thread connection 13, 23.

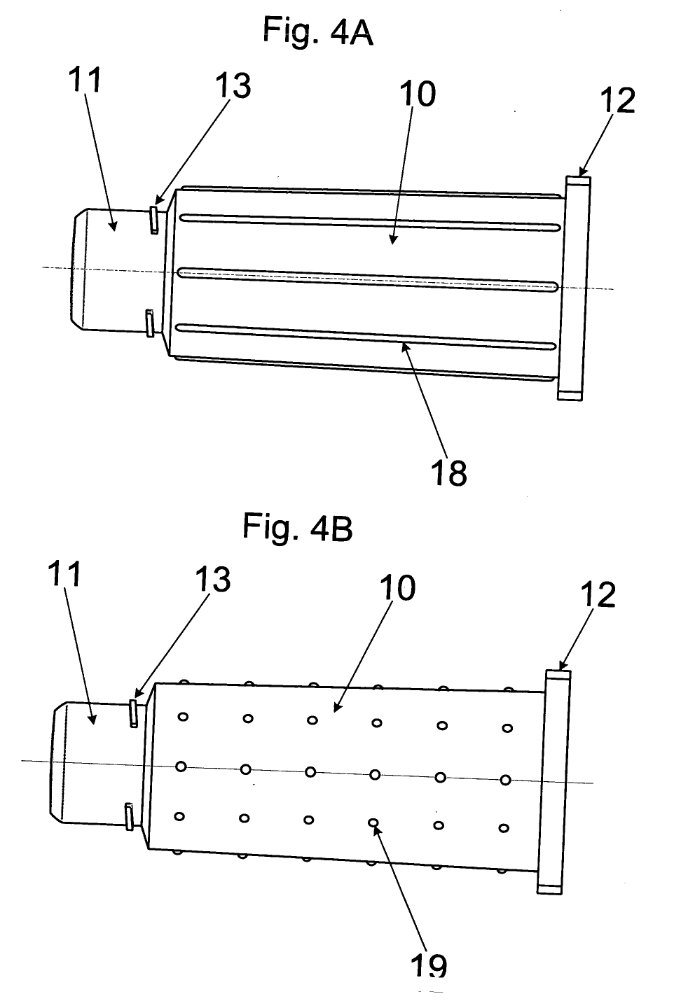

FIG. 4 illustrates how preferably the first cap part 10 can be provided with elevations 18, 19, to locally narrow the space between the first cap part 10, and the second cap part 20, thereby ensuring a good fix of an inserted object 30. Various forms of elevations can be used. FIG. 4A shows an embodiment where a number of longitudinal elevations 18 are provided, and on FIG. 4B the elevations have dot-form.

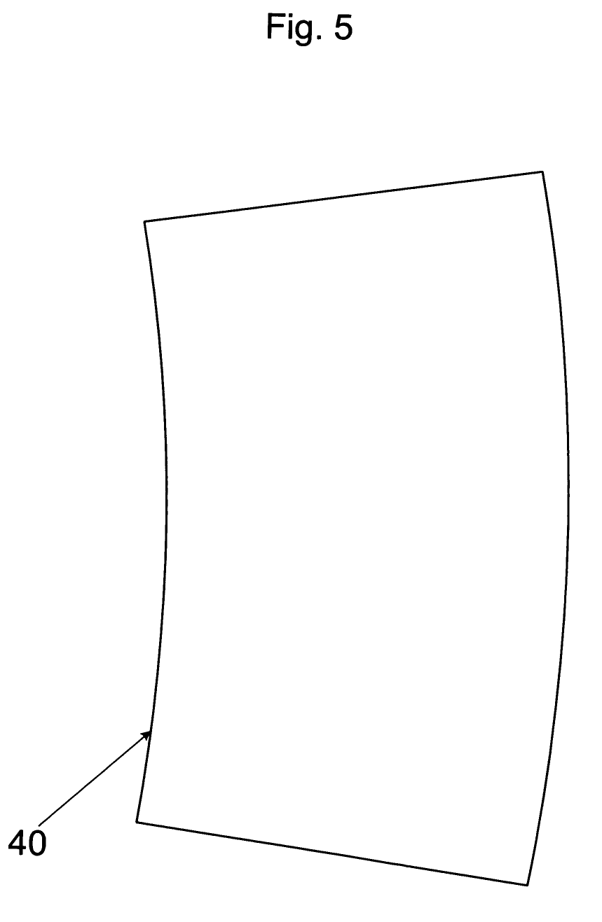

FIG. 5 shows a template 40 for use with the invention. Especially when the cap parts are conical, but also when having other advantageous shapes, it can be difficult to ensure the inserted object 30 has the right dimensions, to fit between the two cap parts 10, 20. A template corresponding the size of the biggest object able to fit between the cap parts with no or only a small overlap is therefore advantageous to use, enabling the user to freely exchange the inserted object 30.

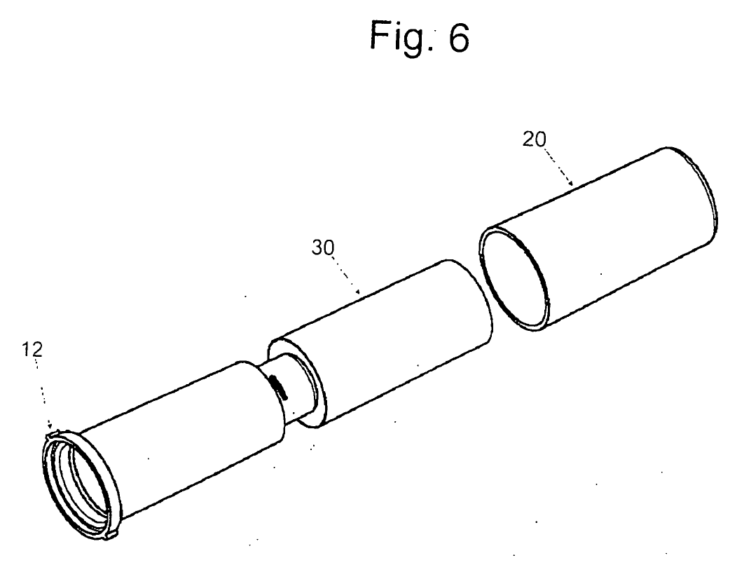

FIG. 6 shows an overall exploded, perspective view of the two cap parts 10, 20 to be assembled with an object 30 to be inserted in between. On this figure the protrusions 12 as well as the opening in the proximal end of the cap can be more clearly seen.

Some preferred embodiments have been shown in the foregoing, but it should be stressed that the invention is not limited to these, but may be embodied in other ways within the subject matter defined in the following claims.

1. A method of providing a safe medical treatment of a patient by motivating said patient to comply with a correct use of a medical device, comprising the steps of
a) providing a medical device with means for visible and interchangeable storing of one or a number of object,
b) providing interchangeable object(s) to the patient initiated by said patient's correct following of given medical treatment instructions.

2. The method according to claim 1 where the objects comprise one or a combination of the following: labels, pictures, patterns, figures, gaming pieces and toys.

3. The method according to claim 1 where the objects comprise information.

4. The method according to claim 3 where the information comprise one or a combination of the following: medication information, injection instructions, inhalation instructions, reminders, emergency telephone numbers, home page addresses, personal addresses, hospital or other emergency addresses, calendars.

5. The method according to claim 1 where the means for interchangeable storing of said object(s) comprise a lock or tamper proof means or tamper evident means.

6. The method according to claim 1 where the objects comprise means for sensing and/or visualizing and/or storing surrounding climatic parameters

7. The method according to claim 1 where said medical treatment instructions comprise one or more of the following: attending scheduled meetings with paramedics, medication instructions, medical device operating instructions,

8. The method according to claim 1 further comprising one or more of the following steps
b) providing a game to be played with said object(s) obtained by the patient
c) providing a bonus system to be initiated when said patient obtains a given number of objects.

9. A cap for releasable attachment to and partial protection of a medical device, characterized in that it comprises two hollow cap parts, the first cap part (10) being designed to fit in the second cap part (20), the two cap parts being designed to releasable interlock to form one assembled unit.

10. A cap according to claim 9, characterized in that the second cap part (20) is at least partly made from a transparent material.

11. A cap according to claim 9, characterized in that a user defined object (30) can be inserted in between the two cap parts (10, 20) when the cap parts are separated, and contained in a gap between the two cap parts when they are assembled.

12. A cap according to claim 9 characterised in that the two hollow cap parts (10, 20) are mechanically interlocked by means of at least one thread (13, 23), snap-lock, bayonet lock, friction means (17), magnets or paste.

13. A cap according to claim 9 characterised by having at least one sealing (16, 17) in between the two cap parts.

14. A cap according to claim 9 characterised in that the first cap part (10) has an axial protrusion (11) at the end opposite to the opening, which protrusion fits into holding means (21) in the second cap part (20), thereby aligning the first cap part (10) concentrically to the second cap part (20) ensuring an even gap between the two cap parts (10, 20) in the full circumference and the full length of the cap.

15. A cap according to claim 9 characterised by the first cap part (10) having at least one radial protrusion (12) at the end with the opening for fitting over the medical device.

16. A cap according to claim 9 characterised in that the first cap part (10) has elevations (18, 19) on the outer side to locally narrow the gap between the two cap parts (10, 20).

17. A cap according to claim 9 characterised in that the outside of the first cap part (10) has a longitudinal supporting edge (14) or a circumferential supporting edge (15) or adhesive material to fixate a user defined object (30) to the first cap part (10).

18. A cap according to claim 16, characterised in that the supporting edge (15) divides the space between the two assembled cap parts into at least two compartments.

19. A template (40) for a cap according to claim 9 characterised in that the outer dimensions of the template corresponds to the largest visual size of a user defined object (30) able to fit between the two cap parts (10, 20).
